(19) United States
(12) Patent Application Publication
(10) Pub. No.: US 2022/0169284 A1
(43) Pub. Date: Jun. 2, 2022

(54) VEHICLE CONTROL DEVICE

(71) Applicant: DENSO CORPORATION, Kariya-city (JP)

(72) Inventor: Noriko KATOH, Kariya-city (JP)

(21) Appl. No.: 17/671,187

(22) Filed: Feb. 14, 2022

Related U.S. Application Data
(63) Continuation of application No. PCT/JP2020/031999, filed on Aug. 25, 2020.

(30) Foreign Application Priority Data
Aug. 30, 2019 (JP) 2019-159015

(51) Int. Cl. B60W 60/00; B60W 40/08

(52) U.S. Cl. CPC B60W 60/0015; B60W 40/08; B60W 2540/01; B60W 2540/22; B60W 2540/225; B60W 60/0013

(57) ABSTRACT

A stress of a passenger is estimated based on biometric information indicating an emotion of the passenger in an autonomous driving vehicle. A stress target is identified as a cause of the stress disposed outside the autonomous driving vehicle based on information on a direction of a line of sight of the passenger when estimating that the passenger has the stress. The vehicle is controlled to increase a safety margin relating to a travel of the vehicle with respect to the stress target.

### CROSS REFERENCE TO RELATED APPLICATION

The present application is a continuation application of International Patent Application No. PCT/JP2020/031999 filed on Aug. 25, 2020, which designated the U.S. and claims the benefit of priority from Japanese Patent Application No. 2019-159015 filed on Aug. 30, 2019. The entire disclosures of all of the above applications are incorporated herein by reference.

### TECHNICAL FIELD

The present disclosure relates to a vehicle control device capable of reducing stress such as anxiety of a vehicle occupant.

### BACKGROUND

Regarding the technique related to the autonomous driving of automobiles (hereinafter referred to as vehicles), in order to realize safe and smooth autonomous driving, the development of image recognition and prediction technique by AI (that is, artificial intelligence) is being promoted. AI is an abbreviation for Artificial Intelligence.

In addition, it is possible to acquire the positions of peripheral targets such as other vehicles and obstacles existing around the vehicle and the attributes of the peripheral targets (that is, the classification of the target).

Further, in recent years, a technique for controlling a vehicle has been developed in consideration of the emotions of a driver or the like who is in an autonomously driven vehicle. For example, a conceivable technique teaches a technique for detecting emotions felt by a driver such as anxiety, a sense of risk, a sense of discomfort, and a degree of tension to control the vehicle in response to the traveling state of an autonomously driven vehicle, for example, the amount of acceleration/deceleration, the timing of acceleration/deceleration, the amount of vibration, and the like.

### SUMMARY

According to an example embodiment, a stress of a passenger is estimated based on biometric information indicating an emotion of the passenger in an autonomous driving vehicle. A stress target is identified as a cause of the stress disposed outside the autonomous driving vehicle based on information on a direction of a line of sight of the passenger when estimating that the passenger has the stress. The vehicle is controlled to increase a safety margin relating to a travel of the vehicle with respect to the stress target.

### BRIEF DESCRIPTION OF THE DRAWINGS

The above and other objects, features and advantages of the present disclosure will become more apparent from the following detailed description made with reference to the accompanying drawings. In the drawings:

FIG. 1 is an explanatory diagram showing an outline of a vehicle system including the vehicle control device of the first embodiment;

FIG. 2 is an explanatory diagram showing the configuration of the vehicle of the first embodiment and its passengers and the like;

FIG. 3 is a block diagram functionally showing the vehicle control device of the first embodiment;

FIG. 4 is a flowchart showing a control process of the first embodiment;

FIG. 5 is an explanatory diagram showing an own vehicle and another vehicle subject to stress in front of the vehicle;

FIG. 6 is an explanatory diagram showing a stressed cliff on the side of the road on which the vehicle travels;

FIG. 7 is an explanatory diagram illustrating the direction of the line of sight with respect to another vehicle which is a stress target reflected in the mirror in the second embodiment;

FIG. 8 is an explanatory diagram illustrating the direction of the line of sight with respect to another vehicle that is a stress target reflected in the electronic mirror; and

FIG. 9 is an explanatory diagram in the case where data is accumulated when there is no peripheral target in the third embodiment.

### DETAILED DESCRIPTION

As a result of detailed examination by the inventor, in the above-mentioned conceivable technique, a difficulty is found such that it may not be possible to deal with stress such as anxiety about factors other than the above emotions, for example, the environment conditions such as another vehicle traveling around the own vehicle while traveling, natural objects around the own vehicle, and the like.

In view of the above points, it is desirable to be able to reduce stress such as anxiety felt by passengers of a traveling vehicle by autonomous driving.

According to an example embodiment, a vehicle control device includes a stress estimation unit, a stress identification unit, and a driving control unit.

The stress estimation unit is configured to estimate whether or not the occupant is stressed based on biometric information indicating the emotions of the occupant in the autonomously driven vehicle.

When it is estimated that the passenger has the stress, the stress identification unit is configured to identify the stress target that is the cause of the stress outside the vehicle based on the information on the direction of the line of sight of the passenger.

The driving control unit is configured to control the vehicle so that the safety margin regarding the travelling of the vehicle is increased with respect to the stress target.

According to an example embodiment, it is possible to reduce stress such as anxiety felt by a passenger of a vehicle traveling by autonomous driving as described below.

Passengers of autonomously driven vehicles may feel stress such as anxiety depending on obstacles such as other vehicles outside the vehicle and the environment such as cliffs near the road. On the other hand, according to an example embodiment, it is estimated whether or not the occupant is stressed based on the biometric information indicating the occupant's feelings, and when it is estimated that the occupant is stressed, the stress target outside the vehicle is identified based on the information on the direction of the person's line of sight. Therefore, it is possible to accurately grasp the stress target.

Then, it has a remarkable effect such that the stress of the occupant is reduced because the running state of the own vehicle is controlled so that the safety margin regarding the running of the vehicle (that is, the own vehicle) on which the occupant is boarded is increased with respect to the stress target.

Hereinafter, exemplary embodiments for implementing the present disclosure will be described with reference to the drawings.

### 1. First Embodiment

[1-1. Overall Configuration]

First, the overall configuration of the vehicle system including the vehicle control device of the first embodiment will be described.

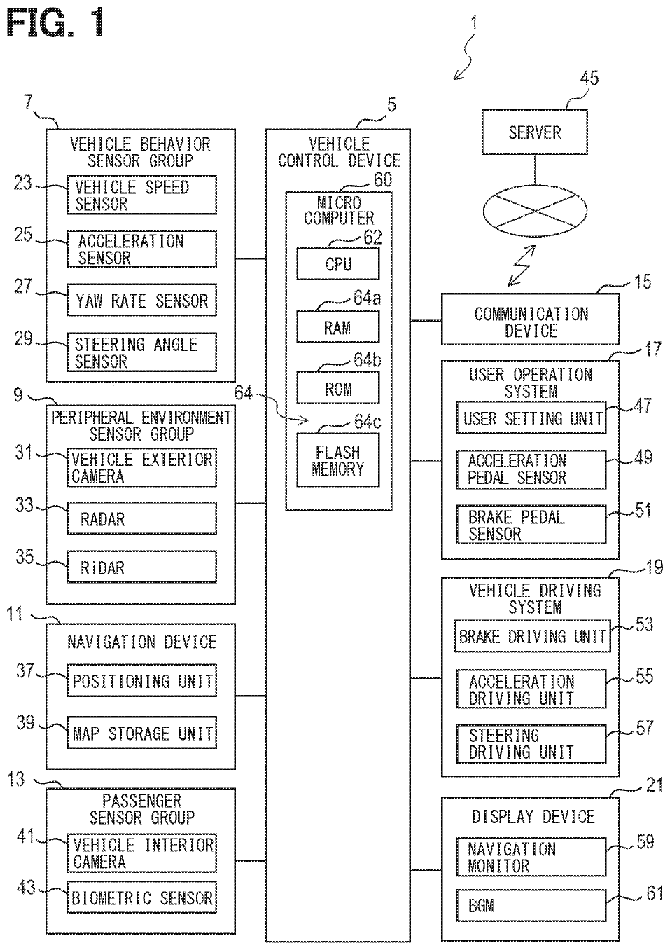

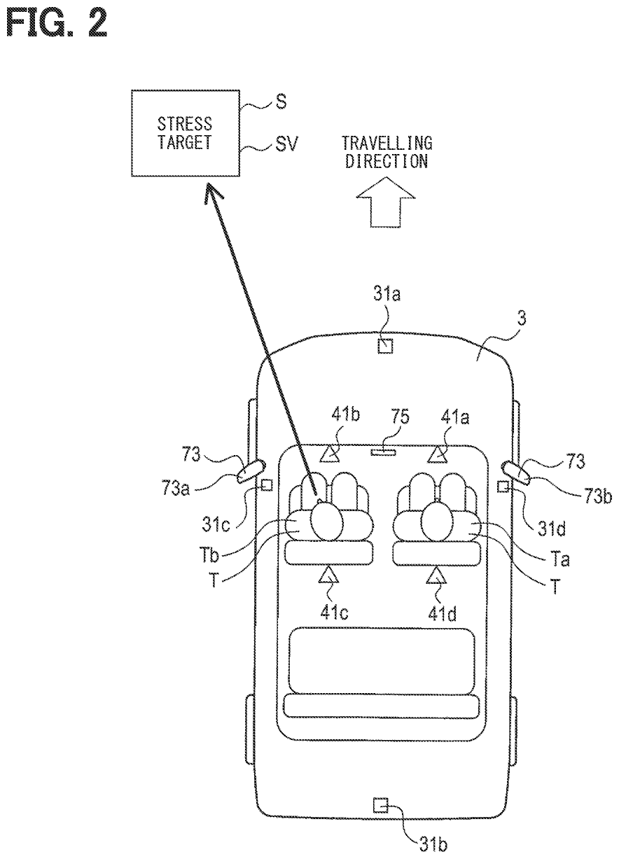

As shown in FIG. 1, the vehicle system 1 is a system mounted on a vehicle 3 which is an automobile (see, for example, FIG. 2), and includes a vehicle control device 5 described later. In the following, the vehicle 3 equipped with the vehicle system 1 may be referred to as a own vehicle (hereinafter, own vehicle) 3.

As shown in FIG. 1, in addition to the vehicle control device 5, the vehicle system 1 may include a vehicle behavior sensor group 7, a peripheral environment sensor group 9, a navigation device 11, a passenger sensor group 13, and a communication device 15, an user operation system 17, a vehicle drive system 19, and a display device 21.

Further, the vehicle 3 can be driven by the driver's operation (that is, driving that is not autonomous driving) and can be driven by autonomous driving. That is, it is possible to operate the vehicle at level 0 and to operate the vehicle by autonomous operation. As the autonomous operation of the vehicle, autonomous operation at level two to five is possible. When referred to as autonomous operation in the present disclosure, it means level 2 or higher autonomous operation.

The level mentioned above includes the standards of the second edition of 2016 set by the Society of Automotive Engineers (that is, SAE) in US.

[1-2. Each Configuration]

Next, each configuration of the vehicle system 1 will be described.

<Vehicle Behavior Sensor Group>

As shown in FIG. 1, the vehicle behavior sensor group 7 is a sensor group that detects the vehicle behavior of the own vehicle 3, and may include a vehicle speed sensor 23, an acceleration sensor 25, a yaw rate sensor 27, and a steering angle sensor 29.

The vehicle speed sensor 23 detects the speed of the own vehicle 3. The acceleration sensor 25 detects the acceleration in the front-rear direction and the acceleration in the vehicle width direction of the own vehicle 3. The yaw rate sensor 27 detects the yaw rate of the own vehicle 3. The steering angle sensor 29 detects the steering angle of the steering wheel of the own vehicle 3. The detection results of these sensors 23 to 29 are output to the vehicle control device 5.

<Peripheral Environment Sensor Group>

The peripheral environment sensor group 9 is a sensor group that detects the surrounding environment of the own vehicle 3, and may include an vehicle exterior camera 31, a radar 33, and a RiDAR 35. RiDAR is an abbreviation for Laser imaging Direction and Ranging.

As the exterior camera 31, as shown in FIG. 2, the front camera 31a for photographing the front side of the own vehicle 3, the rear camera 31b for photographing the rear side of the own vehicle 3, and the left and right side cameras 31c and 31d for photographing the left and right sides of the own vehicle are shown. As the exterior camera 31, a visible light camera, an infrared camera, or the like is used.

The radar 33 uses millimeter waves or the like as radar waves, and detects the distance to a target (that is, a peripheral target) that is an object that reflects the radar wave, the direction in which the target exists, and the like.

The RiDAR35 irradiates the surroundings with a laser beam in a pulse manner, and detects the distance to the target reflecting the laser beam, the direction in which the target exists, and the like based on the reflected light.

Various controls are performed based on the information obtained from the peripheral environment sensor group 9. For example, a target existing around the own vehicle 3 (for example, on a traveling path) is detected by an exterior camera 31, a radar 33, or a LiDAR 35, and target information including the position of the detected target is generated. Then, various controls can be performed based on the generated target information and the like. The peripheral environment sensor group 9 can detect information (for example, distance and direction) related to each target even when a plurality of targets exist. The target information including the position of the target may be generated based on the map information stored in the map storage unit 39 described later.

<Navigation Device>

The navigation device 11 is a device that performs route guidance based on the current position of the own vehicle 3 and map information, and may include a positioning unit 37 and a map storage unit 39.

The positioning unit 37 is a device that generates position information for specifying the current position of the own vehicle 3. The positioning unit 37 includes, for example, a GNSS receiver and a sensor for autonomous navigation such as a gyroscope. The GNSS is an abbreviation of Global Navigation Satellite System.

Map information is stored in the map storage unit 39. The map information is used for route guidance and the like by the navigation device 11.

<Passenger Sensor Group>

The passenger sensor group 13 is a sensor group that detects the state of passengers such as the driver and passengers of the own vehicle 3, and may include an interior camera 41 and a biological sensor 43.

The interior camera 41 is an vehicle compartment camera that captures an image including a face image of a driver or a passenger who is a passenger boarding the own vehicle 3. As the interior camera 41, as shown in FIG. 2, the first camera 41a for photographing the driver's face, the second camera 41b for photographing the passenger's face in the passenger seat, and a third camera 41c and a fourth camera 41d for photographing the passenger's face in the rear seat are used.

An infrared camera can be adopted as the interior camera 41. Thus, the interior camera 41 can photograph a face including an eyeball and detect the direction of the line of sight from the center position of the pupil of the eyeball, for example.

The biological sensor 43 is a sensor that detects biological information indicating the state of the occupant's living body (for example, the state of emotion). Examples of the biological sensor 43 include various sensors that detect various biological information such as heart rate, pulse rate, sweating amount, electrocardiogram, and electroencephalogram. As will be described later, the presence or absence of stress such as anxiety and disgust can be determined from various biological information. For example, when the pulse rate is equal to or higher than a predetermined value and the amount of sweating is equal to or higher than a predetermined value, it may be estimated that anxiety is felt.

<Communication Device>

The communication device 15 is a device capable of transmitting and receiving data to and from the server 45 via the Internet, for example, by using wireless communication.

<User Operation System>

The user operation system 17 is a device for detecting the operation of the driver, and may include a user setting unit 47, an acceleration pedal sensor 49, and a brake pedal sensor 51.

The user setting unit 47 is a manual switch for setting the vehicle speed, for example, when controlling constant speed travel.

The acceleration pedal sensor 49 is a sensor that detects the amount of operation of the acceleration pedal by the driver, and the brake pedal sensor 51 is a sensor that detects the amount of operation of the brake pedal by the driver.

<Vehicle Driving System>

The vehicle driving system 19 is an actuator that drives the vehicle 3, and may include a brake driving unit 53, an acceleration driving unit 55, and a steering driving unit 57.

The brake driving unit 53 is an actuator for applying a brake, and examples thereof include an actuator such as a solenoid valve for adjusting a brake pressure.

The acceleration driving unit 55 is an actuator for accelerating the vehicle 3, and examples thereof include a motor that adjusts the open/closed state of the throttle valve. Further, in the case of an electric vehicle, a motor for rotating the drive wheels can be adopted.

The steering driving unit 57 is an actuator such as a motor that drives the steering wheel.

<Display Device>

Examples of the display device 21 include a navigation monitor 59 that displays map information and the like obtained from the navigation device 11, a rear guide monitor (that is, BGM) 61 that displays an image captured by the rear camera 31b, and the like. BGM is an abbreviation for Back Guide Monitor.

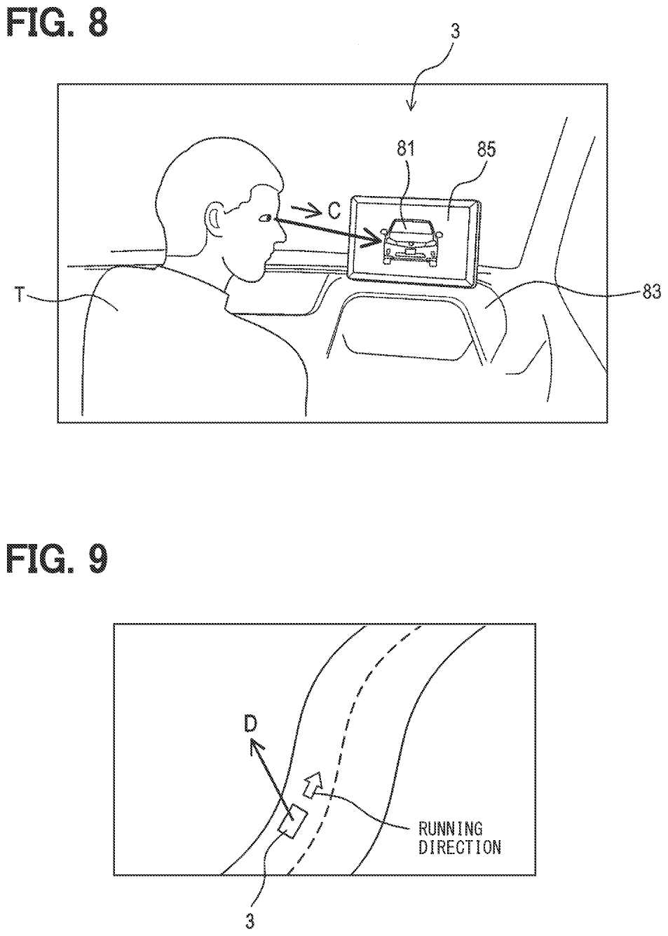

Further, as another display device 21, as shown in FIG. 2, the left door mirror 73a and the right door mirror 73b (that is, the door mirror 73) and the rear-view mirror 75 in the vehicle compartment can be adopted. Further, instead of the left and right door mirrors 73a and 73b and the rear-view mirror 75, an electronic mirror 85 (for example, see FIG. 8) that displays a side or rear image by an LED or the like or a BGM 61 may be adopted. LED is an abbreviation for Light Emitting Diode.

[1-3. Vehicle Control Device]

Next, the vehicle control device 5 will be described.

As shown in FIG. 1, the vehicle control device 5 mainly includes a well-known microcomputer (hereinafter referred to as a microcomputer) 60 having a well-known CPU 62 and a semiconductor memory 64 such as a RAM 64a, a ROM 64b, and a flash memory 64c. Respective functions of the vehicle control device 5 are realized by the CPU 62 executing a program stored in a non-transitory tangible storage medium. In this example, the semiconductor memory 64 corresponds to the non-transitory tangible storage medium for storing a program.

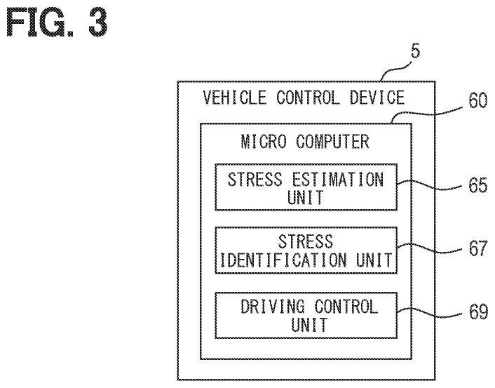

As functionally shown in FIG. 3, the microcomputer 60 of the vehicle control device 5 includes a stress estimation unit 65, a stress identification unit 67, and a driving control unit 69.

The stress estimation unit 65 is configured to estimate whether or not the occupant is stressed based on biometric information indicating the emotions of the occupant in the autonomously driven vehicle 3.

Here, stress refers to a state in which the mind and body are burdened by the environment. In other words, the stress is a negative stress that is not desirable for the passenger. The emotions exhibited by the stress include, for example, so-called negative emotions that are unfavorable to humans, such as anxiety, contempt, disgust, anger, fear, discomfort, tension, and a sense of risk.

These emotions can be estimated, for example, based on a facial image obtained by photographing the passenger's face. In addition, these emotions can be estimated based on signals obtained by various biological sensors 43 that detect the passenger's state (that is, biological state), that is, biological information indicating the biological state. Then, when the above-mentioned emotions are estimated based on the facial image and biological information other than the facial image, it can be estimated that there is stress. The biological information obtained from the facial image is also a kind of biological information. As the biological information, one kind or two or more kinds of biological information can be adopted, and when two or more kinds of biological information are used, it is considered that the accuracy of estimating emotions is improved.

As a technique for estimating emotions from a facial image, various known techniques can be adopted. For example, a facial motion coding system developed by Paul Ekman et al. can be adopted. For example, the technique disclosed in Japanese Patent No. 4101734, JP-2011-117905-A, “Method for measuring human comfort and discomfort by facial image analysis: 2006/9/9, IPSJ Research Report by Hiroyasu Sakamoto et al.” and the like can be adopted and incorporated herein by reference. In addition, emotions can be estimated from facial images using commercially available facial expression estimation software.

In addition, anxiety can be estimated using AI (that is, artificial intelligence) technique, for example, using data obtained by machine learning a large number of facial expressions of anxiety.

Regarding the technique for estimating emotions using facial images, for example, many techniques such as “Emotion estimation technique, https://www.nikkei.com/article/DGXMZO07441910Q6A920C1X90000/”, “Facial muscle movement quantification technique, https://ligare.news/story/aishin_its2018/2/” and the like are disclosed in the Internet websites.

Further, for example, when a biological sensor 43 for detecting biological information such as pulse, heartbeat, sweating, electrocardiogram, and electroencephalogram is provided, based on the information from the biological sensor 43 (that is, biological information), negative emotions such as passenger anxiety can also be detected.

For example, JP-2016-52881-A discloses a method for detecting anxiety, and is incorporated herein by reference. JP-2019-20786-A discloses a method for detecting a sense of risk, and is incorporated herein by reference. JP-2016-7989-A and JP-2014-75008-A disclose a method for detecting the degree of tension (that is, the degree of tension), and are incorporated herein by reference.

When it is estimated that the occupant is stressed, the stress identification unit 67 identifies a stress target S (see, e.g., FIG. 2) as a cause of the stress outside the own vehicle 3, based on the information of the occupant's line-of-sight direction (for example, based on the line-of-sight direction). For example, when a peripheral target is detected ahead of the line of sight, the peripheral target can be regarded as the stress target S.

As a method of detecting the direction of the line of sight, a method using a face image obtained by taking a picture of the above-mentioned interior camera 41 can be adopted. Further, when the passenger sensor group 13 is equipped with an eye camera (that is, an eye mark recorder), the direction of the line of sight can be detected by the eye camera. This eye camera is a device that irradiates the cornea of the human eye with light and detects the direction of the human line of sight and the position of the eye based on the reflected light. The direction of the line of sight can be detected multiple times.

Techniques for detecting the direction of a passenger's line of sight using a facial image are disclosed in, for example, JP-A-2014-7500, JP-A-2012-38106, which are incorporated herein by reference.

Regarding the technique to detect the direction of the line of sight using a face image, for example, many techniques such as “http://moprv.topaz.ne.jp/carrot/oshimag/2001.03/ywata.pdf”, “https://www.eyemark.jp/application-library/driving/” and the like are disclosed in the Internet websites, and are incorporated herein by reference.

The driving control unit 69 controls the vehicle 3 so that the safety margin related to the traveling of the vehicle 3, that is, the safety margin during traveling is increased with respect to the stress target S.

Here, the safety margin related to the traveling of the vehicle 3 is a margin or a leeway provided for ensuring the safety when the vehicle 3 travels. Regarding the control for increasing the safety margin, for example, as described in JP-2019-20786-A, a control for avoiding the stress target S (for example, a control for changing the lane), a control for increasing the distance to the stress target S in the front-rear and/or right-left direction, and the like are adopted. For example, control for increasing the safety margin by adjusting the deceleration timing, deceleration amount, acceleration timing, acceleration amount, and the like to make it difficult to approach the stress target S may be adopted.

[1-4. Control Process]

Next, the control process performed by the vehicle control device 5 will be described.

This control process is a process for increasing the safety margin regarding the running of the vehicle 3 according to the stress of the occupant during autonomous driving. In the following, the safety margin related to the running of the vehicle 3 may be simply referred to as the safety margin of the vehicle 3.

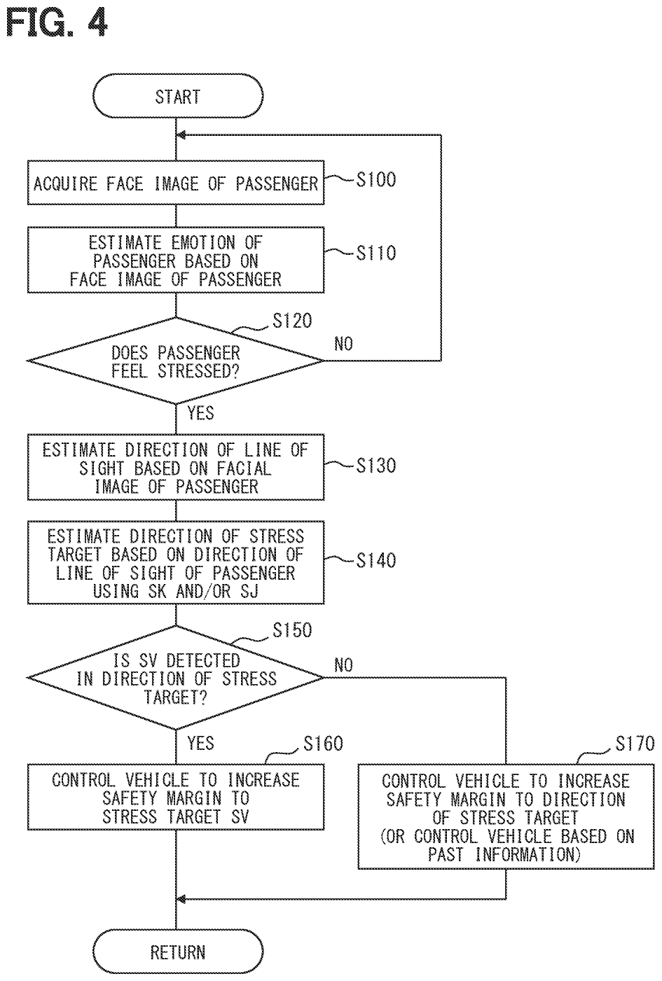

As shown in FIG. 4, in step (hereinafter, S) 100, for example, a face image of a occupant is acquired during autonomous driving of level 2 or higher. For example, the interior camera 41 is used to capture a face image of the passenger, and the face image is input to the vehicle control device 5.

Note that FIG. 2 shows the driver Ta sitting in the driver's seat and the passenger Tb seated in the passenger seat as the passengers T, alternatively, in the following, for the sake of simplification of the explanation, an example of the passenger T is shown as an example of the passenger Tb sitting in the passenger seat may be mentioned.

In the following S110, the emotion indicating the stress of the passenger T is estimated based on the facial image of the passenger T (for example, the passenger Tb). Here, a process for estimating at least one of a person's negative emotions, such as anxiety, contempt, disgust, anger, fear, discomfort, tension, and risk, using the known techniques described above is performed.

Negative emotions may be estimated based on the signal from the biological sensor 43 instead of the facial image. That is, negative emotions may be estimated based on the biological information obtained from the biological sensor 43, for example, based on one type or two or more types of biological information. Further, by combining the face image and the signal from the biological sensor 43, negative emotions can be estimated more accurately.

In the following S120, it is determined whether or not the passenger T is stressed, that is, whether or not the passenger T feels stressed, based on the above-mentioned emotion estimation result. When an affirmative determination is made here, the process proceeds to S130, while if a negative determination is made, the process returns to S100. That is, when it is estimated that the passenger T has negative emotions, it is determined that the passenger T has stress.

In S130, it is presumed that the passenger T has negative emotions, and therefore it is determined that there is stress. Therefore, using the above-mentioned known technique, based on the facial image of the passenger T determined to be stressed, the direction of the line of sight of the passenger T is estimated.

In the following S140, the direction of the stress target S is estimated based on the information on the direction of the line of sight of the passenger T (for example, from the direction of the line of sight). For example, as shown in FIG. 2, when the direction of the line of sight of the passenger T in the passenger seat is the direction of looking out of the vehicle, and the direction of the line of sight is the front left, it is estimated that the stress target S is disposed in front of the left.

Here, since the line of sight of the passenger T is usually not constant, it is not easy to grasp whether or not the direction of the line of sight points to the direction of the stress target S. Therefore, here, the stress target S is specified based on the number of times of viewing and/or the viewing time of the passenger T with respect to the target ahead of the line of sight of the passenger T.

That is, when the passenger T has stress such as anxiety about the stress target S, it is conceivable that the passenger T looks at the stress target S or turns a face to the stress target S many times or stares at a certain direction for a long time. Therefore, in such a case, it is estimated that there is a stress target (that is, a stressor) S that causes emotions such as anxiety in the direction of the line of sight of the passenger T.

For example, when the number of times of looking in a certain direction (that is, the number of times of viewing SK) is equal to or more than a predetermined determination value within a predetermined time, or the total time of looking in a certain direction within a predetermined time (that is, the viewing time SJ) is equal to or greater than a certain determination value, it is estimated that some object in the viewing direction is the stress target S. It may be desirable that the estimation accuracy is further improved when both the number of times of visual recognition and the visual recognition time are satisfied.

Therefore, in this S140, when the above-mentioned conditions of the number of times of visual recognition and/or the visual recognition time are satisfied, it is estimated that the direction in which the line of sight of the passenger T faces is the direction in which the stress target S exists.

In the following S150, it is determined whether or not a peripheral target (for example, see SV in FIG. 2) is detected in the direction of the stress target S. When an affirmative determination is made here, the process proceeds to S160, while if a negative determination is made, the process proceeds to S170.

As a method for detecting the peripheral target SV such as an obstacle existing around the own vehicle 3, for example, the known techniques described in JP-2019-20786-A, JP-2016-52881-A, and JP-2017-166998-A and the like can be adopted. For example, by using the exterior camera 31, the radar 33, the RiDAR35, and the like in the peripheral environment sensor group 9, other vehicles (that is, other automobiles) existing around the own vehicle 3 and obstacles such as stationary objects can be detected as a peripheral target SV.

In S160, since the peripheral target SV is detected in the direction of the stress target S, the peripheral target SV is regarded as the stress target S, and the control is performed to increase the safety margin of the own vehicle 3 with respect to the peripheral target SV. Then, this process is temporarily terminated.

For example, as shown in FIG. 2, when there is another vehicle which is a peripheral target SV corresponding to the stress target S in front of the left side of the own vehicle 3, the speed of the own vehicle 3 may be reduced so as to increase the distance between the own vehicle 3 and the other vehicle in the front-rear direction (that is, the inter-vehicle distance). Alternatively, the position of the own vehicle 3 may be moved to the right side by controlling the steering angle of the own vehicle 3 to the right so that the distance between the own vehicle 3 and the other vehicle in the vehicle width direction increases.

That is, the own vehicle 3 may be controlled so as to avoid another vehicle that is the stress target S. Here, avoidance is to control the vehicle for example, to avoid another vehicle as the stress target S, that is, to increase the distance to the other vehicle. In the case of overtaking, the vehicle approaches another vehicle once, and then, eventually overtakes the other vehicle to increase the distance, which is an example of avoidance.

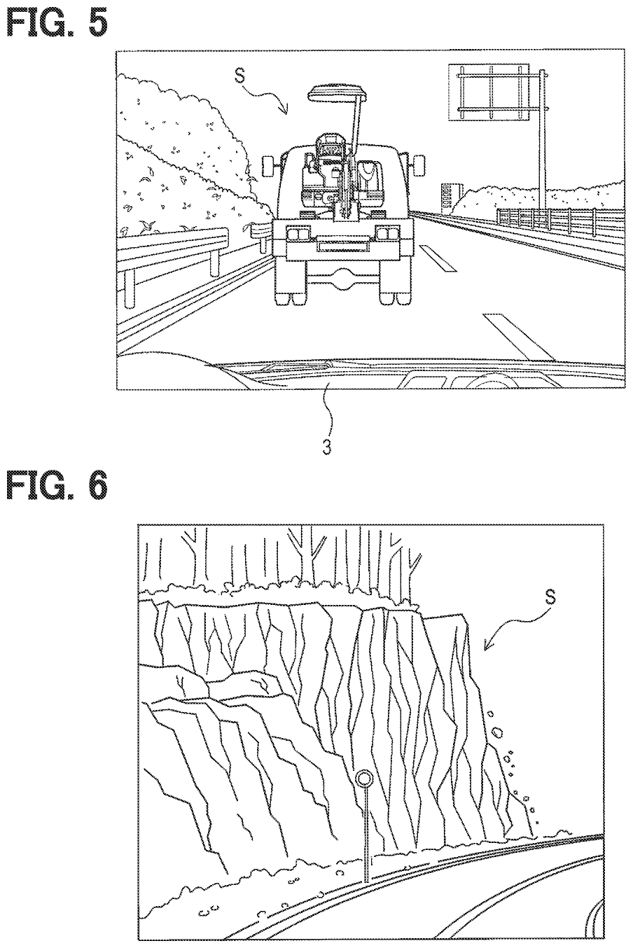

Further, as shown in FIG. 5, when another vehicle that is the stress target S, for example, another vehicle loaded with a large construction machine, is traveling in front of the own vehicle 3 in the same lane, the own vehicle 3 may be decelerated to increase the inter-vehicle distance. On the contrary, when another vehicle which is the stress target S is traveling behind the own vehicle 3, the speed of the own vehicle 3 may be increased to increase the inter-vehicle distance.

Furthermore, when the road has two or more lanes on each side and the own vehicle 3 and the other vehicle are traveling in the same lane, the lane change of the own vehicle 3 may be performed so as to avoid the other vehicle that is the stress target S.

Further, as shown in FIG. 6, when the stress target S is not another vehicle but a dangerous cliff around the road, the course on which the own vehicle 3 travels may be changed so as to move away from the cliff or the like.

On the other hand, in S170, since the peripheral target SV is not detected in the direction of the stress target S, the control for increasing the safety margin of the own vehicle 3 is performed in the direction in which the stress target S may exist, and then, the process ends temporarily.

That is, it is possible to predict that the stress target S is in the direction of the estimated stress target S, and to control the vehicle to increase the safety margin of the own vehicle 3 with respect to the estimated stress target S.

For example, when it is expected that the stress target S is in front of the own vehicle 3, the control may be performed to reduce the speed of the own vehicle 3.

It should be noted that not only when the stress target S is actually detected, but also when the stress target S cannot be detected and the stress target S is predicted, it can be considered that the stress target S is specified.

[1-5. Effects]

In the first embodiment, the following effects can be obtained.

(1a) In the vehicle control device 5 of the first embodiment, it is estimated whether or not the passenger T is stressed based on the biological information indicating the emotion of the passenger T, for example, the biological information obtained from the facial image. Then, when it is estimated that there is stress, the stress target S such as another vehicle outside the own vehicle 3 (that is, outside the vehicle) or the surrounding environment such as a cliff is specified from the direction of the line of sight of the passenger T. Therefore, it is possible to accurately grasp the stress target S.

Then, the running or the like of the own vehicle 3 is controlled so that the safety margin of the own vehicle 3 is increased with respect to the stress target S outside the vehicle. For example, the traveling of the own vehicle 3 is controlled so as to avoid the stress target S. Therefore, there is an effect that the stress of the passenger T is reduced.

As described above, in the first embodiment, even when the passenger T of the vehicle 3 traveling by autonomous driving feels stress such as anxiety due to the stress target S outside the vehicle, it has a remarkable effect that the stress can be reduced.

(1b) In the first embodiment, the stress target S is specified based on the number of times the passenger T is visually recognized and/or the viewing time with respect to the target in the line of sight of the passenger T, so that the stress target S outside the vehicle can be accurately obtained.

(1c) In the first embodiment, when the peripheral target SV is detected by the peripheral environment sensor group 9 in front of the line of sight of the passenger T, the peripheral target SV is regarded as the stress target S, and therefore the position of the stress target S can be recognized with high accuracy.

Therefore, it is possible to suitably control the vehicle to increase the safety margin of the own vehicle 3 with respect to the stress target S.

(1d) In the first embodiment, even if the peripheral target SV is not detected in the line of sight of the stressed passenger T, that is, in the direction in which the stress target S may exist, it is possible to control the vehicle to increase the safety margin of the own vehicle 3 in the direction in which the possibility of existence of the stress target S. Therefore, there is an effect that the stress of the passenger T is reduced.

[1-6. Correspondence of Terms]

In the relationship between the first embodiment and the present disclosure, the vehicle 3 corresponds to an example of a vehicle. The vehicle control device 5 corresponds to an example of the vehicle control device. The stress estimation unit 65 corresponds to an example of the stress estimation unit. The stress identification unit 67 corresponds to an example of the stress identification unit. The driving control unit 69 corresponds to an example of the driving control unit. The peripheral environment sensor group 9 corresponds to an example of the detection unit.

### 2. Second Embodiment

Since the basic configuration of the second embodiment is similar to that of the first embodiment, the differences from the first embodiment will be mainly described below. The same reference signs as in the first embodiment denote the same elements or components, and reference is made to the preceding description.

In the second embodiment, there is a feature in the method of estimating the direction of the line of sight of the passenger T, and this feature will be described.

In the second embodiment, when the display device 21 such as the rearview mirror 75, the door mirrors 73a, 73b, and the BGM 61 is disposed in the line of sight of the passenger T, the object outside the vehicle 3 is regarded as the stress target S which is reflected on the display device 21 ahead of the line of sight. Hereinafter, a specific description will be given.

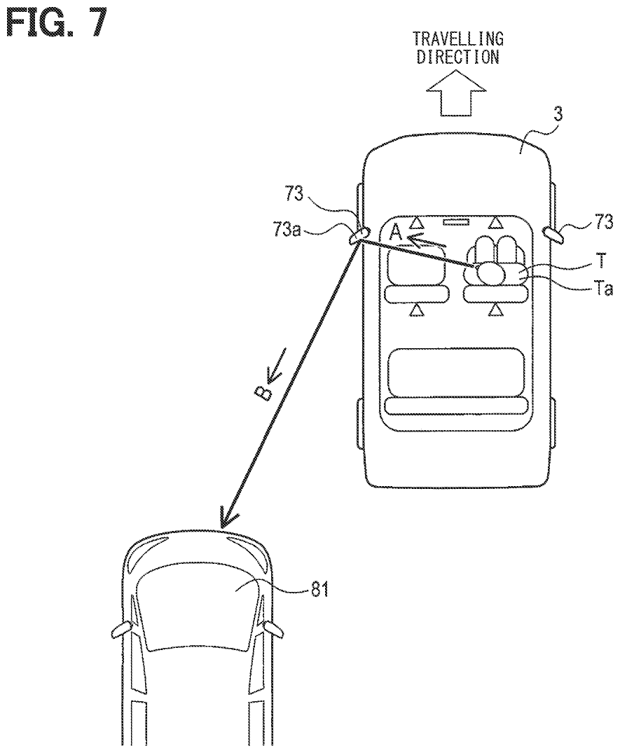

For example, as shown in FIG. 7, when the direction of the line of sight of the passenger T (for example, driver Ta) of the own vehicle 3 is the direction of the left door mirror 73a (for example, the direction of arrow A), in general, the passenger T does not look at the door mirror 73a, but looks at the object on the left rear side (for example, another vehicle 81) reflected on the door mirror 73a.

Therefore, in this case, the direction in which the line of sight is reflected by the door mirror 73a (for example, the direction of arrow B) is set as the direction of the passenger's line of sight.

Since the angle of the mirror of the door mirror 73 is known in advance, if the direction of the arrow A is known from the direction of the line of sight of the passenger T, the direction of the arrow B can be obtained.

Further, as shown in FIG. 8, even when an electronic mirror 85 for displaying an image of the rear of the own vehicle 3 is arranged on the dashboard 83 of the own vehicle 3, for example, an object outside the vehicle (for example, another vehicle 81) reflected in the electronic mirror 85 in front of the line of sight is regarded as the stress target S.

Specifically, when the direction of the line of sight of the passenger T (for example, the driver Ta) of the own vehicle 3 is the direction of the other vehicle 81 on the electronic mirror 85 (for example, the direction of the arrow C), the direction of the line of sight of the passenger T is defined as the direction in which the other vehicle 81 behind the actual own vehicle 3 is being viewed. That is, it is assumed that the direction in which the passenger T is looking at the other vehicle 81 behind from the position of the passenger T (for example, the position of the center of the face).

The direction of the arrow C can be obtained from the face image depending on where the position on the electronic mirror 85 is viewed. Therefore, when the other vehicle 81 is displayed ahead of the line of sight, it can be seen that the passenger T looks at the image of the other vehicle 81.

The second embodiment has the same advantages as those of the first embodiment. Further, in the second embodiment, there is an advantage that the direction of the line of sight can be obtained not only in the direction of the direct line of sight of the passenger T but also when a mirror or a monitor is used.

### Third Embodiment

Since the basic configuration of the third embodiment is similar to that of the first embodiment, the differences from the first embodiment will be mainly described below. The same reference signs as in the first embodiment denote the same elements or components, and reference is made to the preceding description.

In the third embodiment, when the stress target S is specified from the direction of the line of sight of the passenger T and the peripheral target SV is not detected ahead of the line of sight of the passenger T, the information on the direction of the line of sight of the passenger T such as another vehicle 81 accumulated when traveling on the same road in the past is used as the information on the direction of the line of sight. Hereinafter, a specific description will be given.

For example, even if the passenger T feels stress such as anxiety about the environment outside the vehicle ahead of the line of sight, it is conceivable that the peripheral environment sensor group 9 may not detect the peripheral target SV.

For example, as shown in FIG. 9, even if the passenger T of the vehicle (for example, own vehicle) 3 traveling on the road feels that there is a stress target S in a predetermined direction outside the vehicle (for example, in the direction of arrow D) at a certain position or in a certain section of the road, the peripheral environment sensor group 9 may not be able to grasp the stress target S as a peripheral target SV.

In such a case, the communication device 15 of the vehicle 3 is used to provide information on the position or the section of the vehicle 3, information that stress is felt in this position or section, and information on the direction of the line of sight of the passenger T and to transmit to the server 45 on the cloud using the Internet or the like. Even when such information is received from another vehicle 81, the server 45 stores the information in a database.

Therefore, when the vehicle 3 travels near the position or in the section by autonomous driving and the peripheral target of the stress target S is not detected in the line of sight of the passenger T, the vehicle 3 is controlled to suppress the stress of the passenger T by increasing the safety margin of the vehicle 3 based on the past information (that is, the past information), which is stored in the database of the server 45 described above. For example, see S170 in FIG. 4 or the description below.

For example, when the own vehicle 3 travels near the position or in the section, the stress of the passenger T can be appropriately suppressed by reducing the speed of the own vehicle 3 based on the accumulated past information. For example, when there is the accumulated past information for a plurality of vehicles (for example, vehicles 81) that have traveled on the same road in the past, the speed of the own vehicle 3 may be reduced to increase the safety margin of the own vehicle 3. When there is no accumulated past information, the control for increasing the safety margin may not be performed.

The third embodiment has the same advantages as those of the first embodiment. Further, in the third embodiment, when the own vehicle 3 travels on the same road as the road on which the other vehicle 81 has traveled in the past, it has the advantage of being able to control the vehicle to suitably suppress stress by using the accumulated data of the other vehicle 81.

### 4. Other Embodiments

Although an embodiment of the present disclosure has been described above, the present disclosure is not limited to the above-described embodiment, and it is possible to implement various modifications.

(4a) The present disclosure can be applied to vehicles capable of autonomous driving at levels 2 to 5.

(4b) Examples of passengers include drivers, passengers in passenger seats, and passengers in rear seats. Passengers to whom this disclosure applies may or may not be set in advance. The disclosure may be applied to a particular passenger if more than one passengers feel stressed.

(4c) As peripheral targets for stress, other vehicles with unstable driving such as meandering, other vehicles with large or unstable loading conditions, other vehicles with black windows, other cars with certain decorations felt uncomfortable, or the like can be defined. In addition, the environment subject to stress includes cliffs where rock slides such as stones and sand are likely to collapse, steep cliffs, and places where trees cover the road.

(4d) The vehicle control device and the process according to the present disclosure may be achieved by a dedicated computer provided by constituting a processor and a memory programmed to execute one or more functions embodied by a computer program. Alternatively, the vehicle control device and the process according to the present disclosure may be achieved by a dedicated computer provided by constituting a processor with one or more dedicated hardware logic circuits.

Alternatively, the vehicle control device and the process according to the present disclosure may be achieved using one or more dedicated computers constituted by a combination of the processor and the memory programmed to execute one or more functions and the processor with one or more hardware logic circuits. The computer program may also be stored on a computer readable non-transitory tangible recording medium as computer executable instructions.

The technique for realizing the functions of the respective units included in the vehicle control device does not necessarily need to include software, and all of the functions may be realized with the use of one or multiple hardware.

(4e) A plurality of functions of one element in the above embodiment may be implemented by a plurality of elements, or one function of one element may be implemented by a plurality of elements. Further, multiple functions of multiple elements may be implemented by one element, or one function implemented by multiple elements may be implemented by one element. In addition, a part of the configuration of the above embodiment may be omitted. Further, at least part of the configuration of the above-described embodiment may be added to or replaced with the configuration of another embodiment described above.

(4f) The present disclosure can be realized in various forms, in addition to the control apparatus described above, such as a system including the vehicle control device as a component, a program for causing a computer to function as the vehicle control device 1, a non-transitory tangible storage medium such as a semiconductor memory storing the program, or a control method of a vehicle control device.

The controllers and methods described in the present disclosure may be implemented by a special purpose computer created by configuring a memory and a processor programmed to execute one or more particular functions embodied in computer programs. Alternatively, the controllers and methods described in the present disclosure may be implemented by a special purpose computer created by configuring a processor provided by one or more special purpose hardware logic circuits. Alternatively, the controllers and methods described in the present disclosure may be implemented by one or more special purpose computers created by configuring a combination of a memory and a processor programmed to execute one or more particular functions and a processor provided by one or more hardware logic circuits. The computer programs may be stored, as instructions being executed by a computer, in a tangible non-transitory computer-readable medium.

It is noted that a flowchart or the processing of the flowchart in the present application includes sections (also referred to as steps), each of which is represented, for instance, as S100. Further, each section can be divided into several sub-sections while several sections can be combined into a single section. Furthermore, each of thus configured sections can be also referred to as a device, module, or means.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A vehicle control device comprising:
a stress estimation unit configured to estimate whether a passenger has a stress based on biometric information indicating an emotion of the passenger in an autonomous driving vehicle;
a stress identification unit configured to identify a stress target as a cause of the stress disposed outside the autonomous driving vehicle based on information on a direction of a line of sight of the passenger when estimating that the passenger has the stress; and
a driving control unit configured to control the vehicle to increase a safety margin relating to a travel of the vehicle with respect to the stress target.

2. The vehicle control device according to claim 1, wherein:
the vehicle is controlled to avoid the stress target.

3. The vehicle control device according to claim 1, wherein:
the stress target is identified based on at least one of a numerical number of viewing times by the passenger or a viewing period of the passenger with respect to an object along the line of sight of the passenger.

4. The vehicle control device according to claim 1, wherein:
when the line of sight of the passenger is directed to a display device including at least one of a rearview mirror, a door mirror, an electronic mirror, and a rear guide monitor, an object outside the vehicle displayed on the display device, which is ahead of the line of sight, is identified as the stress target.

5. The vehicle control device according to claim 1, wherein:
in a case where the vehicle includes a detection unit for detecting a peripheral target disposed around the vehicle, when the detection unit detects the peripheral target disposed ahead of the line of sight of the passenger, the peripheral target detected ahead of the line of sight is identified as the stress target.

6. The vehicle control device according to claim 1, wherein:
information of a direction of the line of sight stored in a server outside the vehicle for each passenger of a plurality of vehicles that have traveled in a same place previously when estimating that the passenger has the stress is defined as information on the direction of the line of sight when estimating that the passenger has the stress.

7. The vehicle control device according to claim 1, further comprising:
one or more processors; and
a memory coupled to the one or more processors and storing program instructions that when executed by the one or more processors cause the one or more processors to provide at least: the stress estimation unit; the stress identification unit; and a driving control unit.
